(19) United States
(12) Patent Application Publication
(10) Pub. No.: US 2004/0151679 A1
(43) Pub. Date: Aug. 5, 2004

(54) Dissolving agent for melanin

(76) Inventor: Irina M. Mogilevich, Petersburg (RU)

Correspondence Address: HOUSTON ELISEEVA, 4 MILITIA DRIVE, SUITE 4, LEXINGTON, MA, 02421, US

(21) Appl. No.: 10/763,000

(22) Filed: Jan. 22, 2004

Related U.S. Application Data
(63) Continuation of application No. PCT/US02/23822, filed on Jul. 25, 2002.
(60) Provisional application No. 60/344,860, filed on Nov. 7, 2001.
(60) Provisional application No. 60/308,701, filed on Jul. 30, 2001.

(51) Int. Cl. A61K 7/06

(52) U.S. Cl. 424/70.1

(57) ABSTRACT

The invention is related to cosmetology, or, more exactly, to agents for hair activation, hair bleaching, hair coloring and hair conditioning. The invention may found application in the manufacture of cosmetic agents, in cosmetology, in hairdressing and for bleaching and coloring of any type of keratin-containing fibers, for example, eyelashes, natural wool, fur and plumes. The claimed group of inventions is based on the use of perfluoro-organic compounds as solvent for melanin. Emulsions containing perfluoro-organic compounds of the “oil-in-water” type (direct emulsion) and “water-in-oil” type (reverse emulsions) represent the compositions for treating keratin-containing fibers. Emulsions are stabilized by surfactants and contain dispersed particles of specified size, which fact makes it possible for them to penetrate inside a hair. Compositions for hair bleaching and coloring additionally include a bleaching or dyeing agent. The compositions are non-toxic and cause no damage to hair. After a period of 10-20 minutes the compositions produce intensive coloring of keratin-containing fibers that is preserved for a long period of time.

### RELATED APPLICATIONS

This application is a Continuation of International Application Serial Number PCT/US02/23822 filed on Jul. 25, 2002 which claims priority to provisional application serial No. 60/344,860 filed on Nov. 7, 2001 and 60/308,701 filed on Jul. 30, 2001 all of which are incorporated herein by reference in their entirety.

### FIELD OF THE INVENTION

The invention is related to cosmetology, or, more exactly, to agents for hair activation, hair bleaching, hair coloring and hair conditioning. The invention may found application in the manufacture of cosmetic agents, in cosmetology, in hairdressing and for bleaching and coloring of any type of keratin-containing fibers, for example, natural wool, fur and plumes.

The following concepts and acronyms are used in the description:

Hair coloring—changing the tinge or color of hair through the use of chemical agents [H. Willamo. Cosmetic Chemistry (in Russian).—Moscow, Mir, 1990. P. 239].

Keratin—an insoluble though swelling in water fibrillar protein, the main constituent of hair. [The Great Encyclopedic Dictionary (in Russian).—Moscow: Bolshaya Rossiyskaya Entsiklopediya, 1997. P. 520].

Melanin—a high-molecular pigment of black or dark brown color determining the color of the hair. In the hair it is contained mainly in the medium fibrous layer (cortex) while it is absent from the colorless surface scaly layer (cuticle) that reflects light and is responsible for hair shine. This is what distinguishes the natural hair color from the artificial one, when the particles of colorant or pigment are distributed throughout the hair, and the hair often loses its shine because of that. Melanin (or several kinds of same) is present in the grains of pigment inside the hair in the form of melanin-protein complexes. The amount of melanin in the hair of one and the same person may be very different: from 10 ppm to 7% [A. Napolitano et al. Microanalysis of Melanins in Mammalian Hair by Alkaline Hydrogen Peroxide Degradation . . . —J. Invest. Dermatol., 2000, vol. 114, N6, p. 1141-1147].

Perfluoroorganic compounds (PFOC)—organic compounds where all or most of all hydrogen, atoms in the hydrocarbon part are substituted with fluorine [A Concise Chemical Encyclopaedia (in Russian).—Moscow, Sovetskaya Entsiklopediya, 1967, v.5, pp.591-603; B. N. Maksimov et al. Commercial Fluoroorganic Products (in Russian).—St Petersburg: Khimiya, 1996, 520 pp.].

HP—hydrogen peroxide H₂O₂.

AH—ammonium hydroxide (water solution of ammonia, aqua ammonia).

MM—molecular mass of a polymer; dimensions in daltons (Da).

MF-30/40—a PFOC, polyperfluoropropylene glycol with MM of 1500 Da.

PFD—a PFOC, perfluorodecalin.

TPA—tri(perfluoropropyl)amine.

Emulsions—dispersed systems consisting of small droplets of liquid (dispersed phase) distributed in another liquid (dispersion medium). The main types of emulsions are: direct emulsions with droplets of nonpolar liquid in a polar medium (oil-in-water emulsions) and reverse emulsions (water-in-oil emulsions)-[The Great Encyclopedic Dictionary (in Russian).—Moscow: Bolshaya Rossiyskaya Entsiklopediya, 1997. P. 1405].

SAA—surfactant that serves as dispersion stabilizer (in this case, emulsifier).

Perftoran—plasma substitute emulsion with gas-transfer function (based on PFD) allowed for clinical use in Russia.

PL—a SAA, phospholipins of egg lecithin.

PR—a SAA, proxanol, a co-polymer of ethylene oxide and propylene oxide.

Chromoxane—a SAA, potassium perfluoro-4-methyl-3,6-dioxaoctansulfonate.

Thymaxol—a SAA, poly-N,N-dimethylaminoethyl metacrylate.

GPC—gel-penetrating chromatography.

Oxidation dyeing/coloring—hair coloring for which an oxidizer (for example, hydrogen peroxide) is used. Two components enter into composition of oxidation colorant (OC). These are: oxidizer and coloring reagent (“oxidation colorant precursor”—substituted phenols and/or aromatic amines). These two components are mixed together immediately prior to hair coloring, and the mixture in the form of aqueous solution is applied to the hair. Once the oxidizer has penetrated into the inside of a hair, it participates in two processes—namely, oxidation plus chemical destruction of melanin and synthesis (through the use of the precursor of oxidation colorant) of artificial pigments that ensure pre-specified hair color [H. Willamo. Cosmetic Chemistry (in Russian).—Moscow, Mir, 1990. P. 244].

Oxidation colorants (OC) serve as the basis for stable (permanent) color of hair. Such a coloring process is based on the fact that dyeing agent is present in water-soluble form (in the form of “preliminary” coloring agent and penetrates inside the hair where it is oxidized, and in the course of this oxidaton it transforms into a dyeing agent proper which imparts required color to the hair. Following the oxidation the dyeing agent is not soluble in water, owing to which fact it is retained in the hair [H. Willamo. Cosmetic Chemistry (in Russian).—Moscow, Mir, 1990. P. 244].

PPD—p-phenylenediamine, an intermediate product for the oxidation method of hair coloring (oxidation colorant precursor).

AP—p-aminophenol, an intermediate product for the oxidation method of hair coloring (oxidation colorant precursor).

### BACKGROUND OF THE INVENTION

All known hair coloring agents can be divided into two groups [Hair & Hair Deseases,—Ed. by C. E. Orfanos and R. Happle.—Springer-Verlag Berlin, 1989, pp. 887-891]. The first group is meant for surface dyeing of hair. The known agents contain a dissolved or dispersed colorant. The agent in liquid form is applied to the hair and after drying the dye adheres to the hair surface or remains in discontinuities of surface structures. An advantage of this method of changing the hair color is the weak, purely superficial action of the colorant on the hair keratin, a disadvantage—a short life (new hair color is washed out when the hair is washed, or abraded when the hair is brushed) and low covering power of the dye (i.e. the original hair color shows through the layer of applied dye).

Such agents which do not break down the structure of the hair's own pigment (melanin) are usually referred to as color tinting or toning. This group includes color-tinting hair setting lotions, hair ink, color mousses, foams, shampoos and balms. In this list, these cosmetics are listed in the order of their growing resistance to washing out (from 1 to 6 hair wash procedures). Tinting agents can provide any degree of slight change in the hair color (to half a shade), with the exception of bleaching (decolorizing), which up to now was possible only through chemical destruction of melanin [Parfumeriya I Kosmetika (in Russian), 1998, No. 2, pp. 292-293].

The use of tinting agents in combination with vitamins and balms may intensify the original hair color. The intensified, freshened, more vivid effect on the natural hair color is observed as a color change of 0.2-1 shade and in terms of spectroscopy is explained by a higher intensity of the reflected light without change in its spectral composition.

The second group of agents for changing the hair color is aimed at dyeing the hair from within. The hair color can be thus changed both to lighter (bleaching) and darker shades (coloring).

It is known from optics that the color intensity as perceived by human eye, depends on the size of the pigment particles, and this dependence can be described by a curve whose maximum corresponds to a certain range of particle sizes, specific for each color [P. I. Yermilov et al. Pigments and Pigmented Paint & Varnish Materials (in Russian).—Leningrad: Khimiya, 1987, p. 37]. Therefore, with growth of the size of pigment particles, beginning with infinitely small sizes, their reflecting potential will first grow, and then, after their size gets bigger than optimum, decrease. As of today, there are no methods for calculating the reflecting capacity of a conglomerate of non-spherical particles different in size. In the animate nature, there are organisms changing the intensity of their color through aggregation or dispersion of pigment granules in their skin [J. of Evolutionary Biochemistry and Physiology (in Russian), 1993, v.29, No. 5-6, pp. 659-662], [R. I. Garcia et al. Effect of Prostaglandins, 5-bromodeoxyuridine and Ultraviolet Light on Growth and Differentiation of Retinal Pigment Cells in vitro.—In: Pigment Cell, vol.4, p. 177-184], [G. Britton.—Biochemistry of Natural Pigments (in Russian).—Moscow: Mir, 1986, p. 380], [A. M. Castrucci et al. Melanin Concentrating Hormone (MCH) Control of Chrormatophores.—In: Advances in Pigment Cell Research, Ed. by J. T. Bagnara, N.Y., 1988, pp. 547-557]. The diameter of pigment grains of human hair constitutes 0.3-0.7 μm [J. Duchon, Z. Pechan. Biochemie melaninu a melanogenese. Praha, 1964, p.27].

To bleach (whiten) the hair, aqueous solutions of bleaching (whitening) agents—i.e. substances that break down melanin—are introduced into the hair. Oxidizers (such as hydrogen peroxide, persulfates, perborates, and so forth) or reducing agents (such as sulfites, hydrosulfites, thioacids, and the like) may serve as such substances.

Oxidation dyeing represents the most commonly used method for dyeing hair from inside. Colorant components—namely, oxidizer (as a rule, hydrogen peroxide in the presence of aqueous solution of ammonium is used as such) and oxidation colorant precursor—are mixed together, and the mixture is then applied to the hair. Once the oxidizer has penetrated into the inside of a hair, it participates in two processes—namely, oxidation plus destruction of melanin (i.e. mitigation of the original color of the hair) and synthesis of artificial pigments through the use of the precursor of oxidation colorant.

The known agents intended for hair bleaching and hair coloring from inside produce a longer lasting and more intensive hair color than those, which affect only the surface of the hair. However, the agents now in use are aggressive, toxic and, therefore, they are not only damaging to the hair (both keratin and melanin) but may also induce skin allergy [Hair & Hair Diseases,—Ed. by C. E. Orfanos and R. Happle.—Springel-Verlag Berlin, 1989, pp.887-954, pp. 892-898, 927-953; Parfumeriya i Kosmetika (in Russian), 1998, No. 2, pp. 294-298]. In contrast to tinting compositions, this second group of hair color changing agents is referred to as hair dyes in conventional classifications.

Therefore a priority in the process for stable coloring of the hair is the neutralization (partial destruction) of melanin and after that only—formation of a synthetic colorant—i.e. hair coloring proper.

It has been established that the model melanin, on account of its own phenols and carboxyl groups, reacts with bases, thus producing brightly colored (from yellow to black/brown) salts soluble in water (polymeric, oligomeric and monomeric)—[Hair & Hair Diseases,—Ed. by C. E. Orfanos and R. Happle.—Springel-Verlag Berlin, 1989, pp. 166-173; A. Napolitano et al. Microanalysis of Melanin Hair by Alkaline Hydrogen Peroxide Degradation.—J. Invest. Dermatol, 2000, v. 114, N6, pp. 1141-1147]. This is why long-term high-temperature alkaline extraction [I. S. Afanasyeva. Anthropological Aspect of Research into the Pheomelanin Pigment of Hair (in Russian).—Sovetskaya etnografiya, 1981, No. 12, pp. 84-97] or long-term high-temperature extraction with solutions of organic bases, e.g. soluenes—trialkyl ammonium hydroxides [A. Olkawa et al. Quantitative Measurement of Melanin as Tyrosine Equivalents and as Weight of Purified Melanin.—Yale J. of Biology and Medicine, 1973, v.46, p. 500-507] is used to extract melanin pigments from hair, skin or other tissues with the aim of obtaining model melanin for research or diagnostic purposes. However, this approach is not suitable for dyeing “living” hair.

The most common method for the action on the hair melanin, in order to decolorize the hair, is oxidation of melanin with aqueous solution of hydrogen peroxide. This process goes on most efficiently in the presence (up to 1%) of inorganic base (ammonium hydroxide) or organic bases (monoethanolamine, triethylamine) [H. Willamo. Cosmetic Chemistry (in Russian). Moscow: Mir, 1990. P. 239; Hair & Hair Diseases—Ed. by C. E. Orfanos and R. Happle.—Springel—Verlag Berlin, 1989., p.891]. It is considered that alkalization of the oxidizer solution promotes better swelling of keratin in the solution, penetration of the oxidizer into the hair depth through the keratin scales, and higher concentration of peroxide anions (melanin oxidizer proper). The process of in-depth bleaching of hair takes 30 minutes or longer (often this process is repeated several times). This significant disadvantage of the known method can be explained by difficulties in melanin destruction.

The experiments specially staged by the authors showed that dissolution of model melanin obtained from natural materials by ammonium hydroxide takes 5 minutes, and dissolution by aqueous solution of hydrogen peroxide with ammonium hydroxide added-2 minutes (Table 1). Oxidation of the same amount of melanin in the hair using the same agent compositions takes much longer. At least three reasons for this phenomenon can be identified:

it is known that penetration of any reagents from the surface into the hair depth is extremely difficult and takes a long time, because said penetration is accompanied by hair swelling, loosening and breaking down of keratin structures. Increased concentration of hydrogen peroxide (with the value of pH being permanent) speeds the bleaching process up quite insignificantly, but exerts disastrously damaging action on hair. Increased concentration of alkaline agent (growth of pH), with the value of pH being permanent, speeds the bleaching process up quite effectively, but only for pH values greater than 11. However, these values are quite uncomfortable and even hazardous not only for hair quality but also for human scalp health;

in the hair, melanin is chemically bound to the keratin matrix, that is why its dissolution in the hair takes place more slowly and poorly, than that of the model melanin;

removal of the reaction products from the center of the reaction is also difficult. Inside the hair, the capacity of the “reactor” where dissolution and oxidation of melanin takes place, is extremely low. Therefore the resulting reaction products block the access to the fresh oxidizer molecules which have not yet taken part in the reaction, thus preventing full use of hydrogen peroxide in the reaction and full oxidation of melanin in the hair.

Compositions, based on alkaline solutions of hydrogen peroxide have another disadvantage. Reddish-tinted solutions of melanin salts produce an unavoidable residual yellowness in the hair after bleaching procedure is completed [H. Ozeki et al. Chemical characterization of hair melanins in various coat-color mutants of mice.—J. Invest. Dermatol., 1995, v. 105, N3, pp.361-366].

This means that there are objective reasons associated with the character of the process, hair structure, colored salt (ionic) melanin solutions produced, which prevent the acceleration of melanin breakdown and thereby the hair coloring process.

### SUMMARY OF THE INVENTION

Analysis of the known publications, as well as of the hairdressers' practice, shows that a search for high quality, fast working and gentle to the hair coloring and bleaching agents remains an important task.

This problem has been now solved with the claimed new solvent for melanin and a whole range of compositions for changing color of hair and other keratin-containing fibers produced based on this solvent:

a composition for activation of the hair before bleaching or coloring, for color toning, for gray hair cover up (without using dyeing agent) and for hair conditioning;

a composition for bleaching keratin-containing fibers—namely, hair, eyelashes as well as wool, fur and plumes;

a composition for coloring keratin-containing fibers.

The claimed invention is related to the new solvent for melanin and compositions that include this solvent. Treatment of the hair with the claimed compositions performed prior to bleaching and coloring by conventional compositions accelerates and enhances the action of conventional compositions. Penetrating into the depth of hair, the claimed compositions themselves can induce a slight change of color in the absence of oxidizer and colorant, and are non-hazardous (to the same extent as the above-mentioned color tinting agents). At the same time the results of use of the claimed compositions remain there for a long time, like it is characteristic of dyes. Oxidizers and colorants within the claimed compositions produce a more intensive and quick color change in the hair than they would if used without dispersion in the new melanin solvent.

The authors are not aware of other solvents for melanin apart from organic and non-organic bases, or of molecular, not ionic, melanin solutions. The claimed invention proposes that non-ionogenic perfluoroorganic compounds (PFOC) were used for dissolving melanin of keratin-containing fibers. These compounds are non-toxic [B. N. Maksimov et al. Commercial Fluoroorganic Products (in Russian). St Petersburg: Khimiya, 1996, 520 pp.], which is a necessary condition for cosmetics. PFOC produce transparent, non-colored, non-ionized (molecular) solutions of melanin. This application for PFOC was not previously known.

Until now a number of PFOC were known as the base for blood substitutes oxygen carriers [Kuznetsova I. N. et al. Stability of perfluorocarbon emulsions and their compatibility with blood serum.—Art. Cells, Blood Subs. and Immob. Biotech.—1998.—V.262, No. 2, pp. 181-189]: for example, perfluorodecalin in Russia and triperfluoropropylamine in Japan [S. I. Vorobyov et al. A Comparative Study of Certain Perfluorocarbon Emulsions (in Russian). In: Fiziologicheskaya aktivnost ftorsoderzhashchikh soyedineniy.—Pushchino, 1995, pp.33-41]. These compounds are non-toxic and are easily excreted from the organism via lungs [G. R. Ivanitsky. Biophysical Fundamentals for Creation of Perfluorocarbon Media and Gas-carrying Blood Substitutes (in Russian). In: Perftororganicheskiye soyedineniya v biologii i meditsine.—Pushchino, 2001, p. 17.].

PFOC ability to transport oxygen is made use of in cosmetics also, for instance, in manufacturing creams, ointments and shampoos out of direct water emulsions of PFOC [U. U. Akhsyanov, O. E. Oksinoyd, L. A. Makhlis. Akvaftem. Microemulsion of Perfluoroorganic Compounds—a Superefficient Oxygen Carrier (in Russian).—Moscow, 1999, 18 pp.]. The action of these creams and ointments is based on the delivery of gases or physiologically active substances (dissolved in PFOC) to the skin or scalp via the treatment of skin with direct PFOC emulsions [U.S. Pat. No. 5,851,544, C. Penska et al., Cosmetic Skin or Hair Compositions Containing Fluorocarbons Infused Carbon Dioxide, Dec. 22, 1998, Chesebrough-Pond's USA Co.; U.S. Pat. No. 4,569,784, R. E. Moore, Preparation of a gel having gas transporting capability, Feb. 11, 1986, Adamantech, Inc.].

It is also known [U.S. Pat. No. 5,641,509, U. Gross et al., Preparation for Topical Use, Apr. 30, 1996, Lancaster Group AG, DE] that natural or synthetic melanin introduced into skin by loaded liposomes (0.2-3 μm) based on phospholipins (PL) or their modifiers serves as UV-protector. At that the increase in the concentration of melanin in skin is attained via solubilization of melanin not in PFOC, but in a mixture of PFOC either with linear hydrocarbons or with vegetable oils. Said hydrocarbons or oils make it possible to increase the loading of liposomes with melanin. The concentration of melanin inside liposomes may reach 1 g per 1 ml of liquid phase or 50% w/w with regard to PFOC density. The experiments specially staged by the authors of the invention claimed herein showed that the known invention deals with dispersion of melanin in PFOC, but not with a solution, because the maximum concentration of melanin in a solution can not (depending on molecular weight of polymeric melanin) exceed the value 1-2% w/w (see Table 1). The task of the known invention is not to completely dissolve melanin, because this would contradict the main goal of the invention—to protect skin against UV radiation by introducing exogenous melanin with the structure preserved to the maximum degree possible and with simultaneous minimal duration of existence of melanin carriers in an organism. Therefore, the authors of the known invention did not observed (and, hence did not described) the original property of solutions of melanin in PFOC—i.e. transparency and achromatism—found out by the authors of invention claimed herein. The fact that PFOC was selected as a in the known invention is mainly determined by their non-toxicity and short period required to remove them from skin tissues.

In the claimed invention a new application of PFOC is claimed—that of a solvent for melanin of keratin-containing fibers. The variety of PFOC includes perfluorinated hydrocarbons (e.g. perfluorodecalin, perfluorinated bromoctyl bromide, 1,2-dibromtetrafluoroethane), perfluorinated tertiary amines (e.g. tri(perfluoropropyl)amine, dimethyl cyclohexyl amine), perfluorinated ethers (e.g. polyperfluoropropylene glycol), perfluorinated alcohols and ketones or PFOC mixtures.

In comparison to other solvents, the above-mentioned compounds easily (in no longer than 30 s) dissolve model melanin, producing colorless solutions (see examples 10-14 in Table 1). At the same time PFOC don't cause any changes in color or quality of hair even in the case when hair are treated at elevated temperature (up to 60° C.) for many hours. A carrier is necessary to facilitate the introduction of the claimed solvent—a PFOC—into the hair to the location of pigment grains.

TABLE 1
The rate of melanin dissolution and color of some solutions of melanin
extracted from the mycelium and spores of the fungus Aspergillus niger*.

| | | Toxicity** | | | |
|---|---|---|---|---|---|
| | | (maximum | | | |
| | | allowed | | | |
| | | concentration | | | |
| | | in the air in the | Time of | pH of the | Color of the |
| No | Solvent | working area) | dissolution | solution | solution |
| 1 | 0.01 N solution of | — | Not dissolved in 5 | 3.5 | |
| | acetic acid | | hours | | |
| 2 | 4% solution of HP | 1.4 mg/m³ | Not dissolved in 5 | 3.5 | |
| | | [1, v.1, p.32] | hours | | |
| 3 | Triethylamine | 10 mg/m³ | Not dissolved in 5 | 8 | |
| | | [1, v.2, p.522] | hours | | |
| 4 | 0.01 N sodium | — | 6 minutes | 9.5 | Yellow- |
| | hydroxide | | | | brown |
| 5 | 0.01% ammonia | 20 mg/m³ | 5 minutes | 10.5 | -”- |
| | | [1, v.1, p.122] | | | |
| 6 | Monoethanol- | >10 mg/m³ | 2 minutes | 10 | -”- |
| | amine | [1, v.2, p.533] | | | |
| 7 | Mixture of 4% HP | | 2 minutes | 9.5 | -”- |
| | and 0.02% | | | | |
| | ammonia | | | | |
| 8 | 1,2- | 1000 mg/m³ | 5 minutes | 7 | Colorless |
| | dibromtetrafluoroethane | [2, p.246] | | | |
| 9 | Perfluoro-1,3- | >10 mg/m³ | 3 minutes | 7 | Lemon |
| | dimethyl- | [1, v.2, p.533] | | | yellow |
| | cyclohexylamine | | | | |
| 10 | Polyperfluoropropylene | Nonvolatile | 30 seconds | 7 | Colorless |
| | glycol with | | | | |
| | MM 1500 Da (MF- | | | | |
| | 30\40) | | | | |
| 11 | Polyperfluoropropylene | Nonvolatile | 15 seconds | 7 | Colorless |
| | glycol with | | | | |
| | MM 3000 Da | | | | |
| 12 | Tri(perfluoropropyl) | 500 mg/m³ | 30 seconds | 7 | Colorless |
| | amine (TPA) | [2, p.410] | | | |
| 13 | Perfluorodecalin | — | 30 seconds | 7 | Colorless |
| | (PFD) | | | | |

*Based on data of gas-permeation chromatography and component analysis, the model melanin is a low-molecular fraction (with MM being equal to 39000 Da) of poly-5,6-dihydroxyindol-2-carboxyl acid; the concentration of melanin solution in PFOC is equal to 1 mg/ml or 0.05% w/w;
**Information on toxicity is obtained from:
[1] Hazardous Substances in Industry (in Russian)./N. V. Lazarev (ed.).-Moscow: Khimiya, 1971, vols.1 and 2.
[2] Commercial Fluoroorganic Products (in Russian)./B. N. Maksimov et al. (ed.). St Petersburg: Khimiya, 1996.

It is known that in addition to melanin, loaded liposomes are used for the purpose of introducing other loadings (i.e. vitamins and medications) into the scalp and hair follicles [U.S. Pat. No. 5,641,508; Li et al.; “Method for delivering melanin to hair follicles”; Jun. 24, 1997], [U.S. Pat. No. 5,436,010; Lau et al.; “Hair penetrant and carrier”, Jul. 30, 1993; U.S. Pat. No. 5,641,508; Li et al.; “Method for delivering melanin to hair follicles”; Jun. 24, 1997. U.S. Pat. No. 6,284,234. S. M. Niemiec. Topical delivery systems for active agents. Sep. 4, 2001].

The known ceramide compositions (water-insoluble capsule loaded with reagents in water) for hair perming and hair bleaching having reduced hair damaging action [U.S. Pat. No. 6,312,674; M. Maubru et al. “Oxidizing composition and novel method for perming or bleaching hair”, Nov. 6, 2001] are structurally arranged almost in the similar manner.

The use of direct or reverse emulsions represents another known method for the structural arrangement of dispersed reagents to be used for hair treatment.

For example, the known conditioner based on polydimethyl-siloxane is used in the form of aqueous suspension (size of particles from 0.45 to 2 μm) swollen in water-insoluble solvent of polymer particles [U.S. Pat. No. 6,355,233; W. R. Bergman et al: “Hair care compositions and method for depositing swollen polymer particles onto hair”, Mar. 12, 2002].

Reverse emulsions of water-soluble bleaching and coloring reagents (size of particles from 0.01 to 0.15 μm), which enable one to reduce the time of bleaching and oxidation coloring to 5 minutes, are known [U.S. Pat. No. 6,315,989 S. Narasimhan et al. Water-in-oil microemulsion peroxide compositions for use in coloring hair and related methods. Nov. 13, 2001. and U.S. Pat. No. 6,238,653 S. Narasimhan et al Liquid crystalline peroxide compositions and methods for coloring and/or bleaching hair. May 29, 2001]. However, the authors use extremely high concentrations of alkaline agents (ammonia and monoethanolamine) [the calculation made according to the formulations indicated by the author shows that the level of pH is over 12], which fact results both in the reduction of process duration and in highly damaging action on hair.

Known reverse emulsions containing strongly acidic aqueous media (with sulfuric or phosphoric acid) and coloring and bleaching reagents are also characterized by strong damaging action on hair [U.S. Pat. No. 6,274,126. G. Newell et al. Composition for lightening and highlightining hair. Aug. 14, 2001]. The authors propose adding special conditioners to emulsion composition for softening the hair and reducing hair brittleness.

The claimed invention proposes that PFOC were introduced into the hair in the form of emulsion with water. Depending on the method of preparation, it might be either oil-in-water emulsion (direct emulsion) or water-in-oil emulsion (reverse emulsion). The emulsions are stabilized with surfactants and contain dispersed particles of a certain size, allowing them to penetrate inside the hair and break down in locations where the pigment is accumulated, releasing the content of the dispersed phase.

The authors have found out experimentally that treatment of hair by direct aqueous emulsions of PFOC claimed herein results in the following four effects:

activation of the subsequent hairdressing procedure—for example, hair bleaching or dyeing (Table 2),

slight (to a shade) change in hair color (Table 3),

gray cover up without using any coloring agent (Table 4),

conditioning of hair (Table 5).

The interaction of PFOC with melanin granules inside a hair results in the enhancement of hair brightness and freshening of hair color. These effects manifest themselves as melanin granules dissolve inside a hair. Thus, it was established that direct aqueous emulsion of PFOC has a unique property—not only to dissolve melanin, but also to change the intensity of natural hair color (by 0.5-1 shade) in the absence of dyeing agent. These two properties of the PFOC-based emulsion—to dissolve melanin and to modify the hair color at that—can be used in two aspects, as proposed by the authors of the invention.

Firstly, using the emulsion makes it possible to dissolve melanin in the hair to “neutralize” it and to facilitate the delivery of reagents to it. This provides for the preparation of the hair for further, in-depth, bleaching and/or coloring, serving as a non-traumatic first preparatory stage, mentioned above in the description of traditional means and methods of hair coloring. This means that such emulsion represents a universal activator for the bleaching and coloring compositions. Examples of using direct water emulsions of the above-mentioned PFOC for activation of the subsequent hair bleaching with HP are given in Table 2.

TABLE 2
Effect of activation by direct aqueous emulsion of PFD
(perfluorodecalin) on the rate and depth of hair bleaching with HP*.

| | Content of HP in | | | | | | |
|---|---|---|---|---|---|---|---|
| No. of | a bleaching | Activation | | | Bleaching, shade/time | | |
| example | composition | t, ° C. | τ, min | Hair color | 10 min | 30 min | 60 min |
| 14 | 6% | | 0 | Dark Brown-5 | 1.3 | 1.3 | 1.7 |
| 15 | | 40 | 2 × 30 | | 1.5 | 2.0 | 2.0 |
| 16 | | | 0 | Light Broun-2 | 0.1 | 0.5 | 0.6 |
| 17 | | 40 | 2 × 30 | | 2.0 | 2.0 | 2.3 |
| 18 | | 30 | 20 | | 0.6 | 0.9 | 1.2 |
| 19 | 4% | | 0 | Dark Brown-5 | 1.0 | 1.7 | 2.0 |
| 20 | | 40 | 2 × 30 | | 1.2 | 2.0 | 2.3 |
| 21 | | | 0 | Light Brown-2 | 0.2 | 0.3 | 0.4 |
| 22 | | 40 | 2 × 30 | | 2.5 | 2.7 | 3.0 |
| 23 | | 30 | 20 | | 0.4 | 1.0 | 1.5 |

*Emulsion composition: PFD (perfluorodecalin) - 27%, surfactant - PR (proxanol) - 4%, water - 69%; bleaching was conducted at a temperature of 30° C.

Secondly, even in the absence of dyeing agents a direct aqueous emulsion of PFOC itself can serve as hair tinting composition enabling one to change the natural color of hair by 0.5-1 shades (see Table 3). In this case a direct aqueous emulsion of PFOC demonstrates a conditioning effect: following the treatment with such emulsion for a period of no less than 20 minutes, the hair acquire shine, volume and slight waviness (Table 5).

TABLE 3
Tinting of hair with direct aqueous emulsions of PFOC at a
temperature of 30-40° C.

| | | | | | | | Intensification |
|---|---|---|---|---|---|---|---|
| | | PFOC, | | | | Total time | of color |
| No. of | | % by | | SAA, % | Hair | of contact, | (darkening, |
| example | PFOC | mass | SAA | by mass | sample | hours | shade |
| 24 | PFD | 14.5 | PL | 1 | Black-2 | 0.5 | 0.5 |
| | (in the presence | | | | | 0.25 | 0.5 |
| | of AH) | | | | | | |
| 25 | TPA | 54 | Chromoxane | 0.05 | Black-2 | 0.5 | 0.5 |
| | (in the presence | | | | | 0.25 | 0.5 |
| | of AH) | | | | | | |
| 26 | MF-30/40 | 28 | Chromoxane | 0.05 | Black-2 | 0.5 | 0.3 |
| | (in the presence | | | | | 0.25 | 0.3 |
| | of AH) | | | | | | |
| 27 | PFD | 14 | PL | 1 | Blond-2 | 0.5 | 0.4 |
| | (in the presence | | | | | | |
| | of AH) | | | | | | |
| 28 | PFD | 14 | PL | 1 | Blond-2 | 3 × 0.5 | 0.8 |
| | (in the presence | | | | | | |
| | of AH) | | | | | | |
| 29 | PFD | 14 | PL | 1 | Blond-2 | 0.5 | 0.6 |
| | | | | | | 2 × 0.5 | 0.3 |
| 30 | PFD | 56 | PL | 1 | Blond-2 | 0.5 | 1.1 |
| | | | | | | 2 × 0.5 | 0.6 |

The effect of the enhancement of own hair color (color freshening) and conditioning effect are manifested most dramatically when using compositions on hair streaked with gray (Tables 4 and 5). Initially dull and “dead” hair becomes bright; a sample seems to be darkened by an entire shade because gray hair acquire yellowish color (if non-gray hair are dark). This effect is not observed after using known tinting compositions (application of certain tinting compositions is not recommended for hair that become grayish—for example, tinting foam mousse Mouvanance Douceur, harmless dye Mouvance Nature and liquid cream dye Mouvanance Couleur produced by Yves Rocher [Parfumeriya I kosmetika (in Russian), 1998, No. 2, pp. 292-293]).

Another method for exerting action on natural color of hair is known. This method is characterized by local application (on scalp, on hairy areas of human skin) of systemic preparations that suppress or induce melanogenesis bodysections [U.S. Pat. No. 5,958,433. J-T. Simonnet. Stable dispersion of water-immersible phase, in an aqueous phase by means of vesicles based on silicone surfactant. Sept. 28, 1999. U.S. Pat. No. 6,187,325 P. Pelletier et al. Use of at least one extract of rosacea of the genus Sanguisorba officinalis for promoting pigmentation of the skin and/or the body hair and/or the cranial hair. Feb. 13, 2001. U.S. Pat. No. 6,063,389. V. Chevalier et al. Composition for depigmenting or bleaching mammalian skin containing L-2-oxothiazolidine-4-carboxylic acid and a polyol. May 16, 2000. U.S. Pat. No. 6,228,350. R. Toloup et al. Method of depigmentng and/or bleaching skin and/or body hair or head hair. May 8, 2001. U.S. Pat. No. 6,326,014. R. Toloup et al. Use of oxamate derivatives as depigmenting agents. Dec. 4, 2001. U.S. Pat. No. 6,348,204. P. Touzan. Cosmetic or dermatological composition containing at least one extract of mulberry, at least one extract of skullcap and at least one salicylic acid derivative. Feb. 19, 2002]. All above-indicated compositions represent direct emulsions of oils that contain said preparations. At that, it is suggested that oligomer perfluorated polyethers were used in addition to other non-toxic and easily removed from organism oils. Besides hair color change, systemic preparations also change the color of skin. However, the main disadvantage of these preparations consists in significant “interference” in organism metabolism with the aim to attain temporary cosmetic effect.

As can be seen from Table 5, the conditioning effect attained in the presence of direct emulsions of PFOC differs both qualitatively and in terms of duration of action from the effect attained after treating hair with solutions of surfactants (SAA)—stabilizers of direct emulsions.

The known conditioning composition with known conditioning oils (this composition contains up to 35% of surfactant (SAA) and from 0.1 to 30 ppm of perfluorated polyether with MM being within 500-62 000 Da) makes the hair lustrous than the same composition without micro-additives of PFOC does [U.S. Pat. No. 5,451,395 A. M. Murray et al. Hair Treatment Composition. Sept. 19, 1995.]. At the same time, the increase in the content of polyether PFOC to 1% results in the reverse effect—the hair become more dull and greasy. Based on data provided by the authors, from 0.3 to 30 μm of perfluorated polyester gets on the hair of human's head when using a composition with PFOC micro-additives. The average amount of melanin pigment in these hair is from 5 mg [A. Napolitano et al. Microanalisis of Melanins in Mammalian Hair by Alkaline Hidrogen Peroxide Degradation.—J. Invest. Dermatolog., 2000, vol. 114, N6, p. 1141-1147] to 3.5 g [S. Ito et al. Quantitative Analisis of Eumelanin and Pheomelanin in Hair and Melanomas. J. Invest. Dermatol., 1983, vol.80, N4, pp.268-272], which fact completely excludes the possibility that PFOC “acts” as solvent for melanin in said composition. Most likely, a micro-additive of PFOC in this case modifies the action of SAA, and, possibly, the size of droplets of conditioning oil, which results in surface conditioning.

TABLE 4
Covering gray hair up as a result of interaction between hair and
direct aqueous emulsions of PFD at a temperature of 30-40° C.

| No. | | | | | | Total time | |
|---|---|---|---|---|---|---|---|
| of | | PFD, % | | SAA, % | | of contact, | Darkening |
| example | pH | by mass | SAA | by mass | Hair sample | hours | shade |
| 31 | 10 | 14 | PL | 1 | Black-4 | 0.5 | 0.3 |
| | | | | | 5% of gray hair | 0.25 | 0.3 |
| 32 | 7 | 56 | PL | 1 | Black-5 | 0.35 | 0.9 |
| | | | | | 50% of gray hair | 0.7 | 0.9 |
| | | | | | | 2 × 0.5 | 1.1 |
| 33 | 7 | 14 | Proxanol | 1 | Black-5 | 0.35 | 0.5 |
| | | | | | 50% of gray hair | 0.7 | 0.5 |
| 34 | 7 | 27 | Proxanol | 4 | Black-5 | 0.35 | 0.4 |
| | | | | | 50% of gray hair | 0.7 | 0.5 |
| | | | | | | 2 × 0.5 | 1 |
| 35 | 7 | 14 | PL | 1 | Black-5 | 0.35 | 0.6 |
| | | | | | 50% of gray hair | 0.7 | 0.6 |
| 36 | 7 | 56 | PL | 1 | Light Brown-1 | 0.5 | 0.5 |
| | | | | | 30% of gray hair | 2 × 0.5 | 0.5 |

TABLE 5
Conditioning of hair by direct aqueous emulsions of PFOC at a
temperature of 30-40° C.

| | | | Action of shampoo, number | |
|---|---|---|---|---|
| | Hair | | of washing sessions | |
| No. of | | Description of hair | Initial color is | Dryness is |
| example | Treatment type | condition | restored | observed |
| 37 | Initial | Coarse auburn hair (over 50% | — | 2 |
| | | of gray hair) | | |
| 38 | Treated with SAA | Effect of hair | 2 | 1 |
| | (PL) solution | straightening without | | |
| | | making the hair heavier | | |
| 39 | Treated with PFOC | Silky hair - i.e. lustrous | 6 | 5 |
| | emulsion, stabilized | smooth hair | | |
| | with phospholipins | | | |
| 40 | Initial | Wavy light-auburn hair | — | 1 |
| | | with an effect of “dead” | | |
| | | hair | | |
| 41 | Treated with SAA | Enhancement of hair | 1 | 2 |
| | (PL) solution | luster and hair | | |
| | | moistening effect | | |
| 42 | Treated with PFOC | Silky hair - i.e. lustrous | 5 | 5 |
| | emulsion, stabilized | smooth hair | | |
| | with phospholipins | | | |
| 43 | Initial | Coarse auburn hair (over | — | 2 |
| | | 50% of gray hair) | | |
| 44 | Treated with SAA | Conventional | 1 | 2 |
| | (proxanol) solution | conditioning | | |
| | | accompanied by the fact | | |
| | | that the hair are made | | |
| | | heavier | | |
| 45 | Treated with PFOC | Enhancement of hair | 6 | 5 |
| | emulsion, stabilized | luster, straightening of | | |
| | with PR | gray springy hair | | |
| 46 | Initial | Wavy light-auburn hair | — | 1 |
| | | with an effect of “dead” | | |
| | | hair | | |
| 47 | Treated with SAA | Effect of hair | 2 | 3 |
| | (proxanol) solution | straightening | | |
| 48 | Treated with PFOC | Enhancement of hair | 5 | 5 |
| | emulsion, stabilized | luster, the dryness of hair | | |
| | with PR | ends has disappeared | | |

The identified properties of direct aqueous emulsions of PFOC (that manifest themselves in the course of hair treatment) served as a basis for the second invention of the claimed group of inventions—namely, a composition for activating the hair prior to bleaching or coloring, for hair toning, for covering gray hair without using dyeing agent and for conditioning the hair. The claimed composition is a direct aqueous emulsion of PFOC, of “oil-in-water” type, with particles of average size of no greater than 0.5 μm, predominantly 0.04-0.3 μm, stabilized with surfactants. The proportion of the dispersed phase of PFOC in the said composition is from 2 to 95% by mass, of surfactants—from 0.01 to 10%. Any compound from the above-listed PFOC range (melanin solvents) may be used as PFOC. It is possible to use polymer water-insoluble SAA (phospholipines), water-soluble SAA (thymaxol, proxanol) and low-molecular water-soluble ionogenic SAA (chromoxane) as surfactants (SAA). The value of pH of the dispersion medium (water) is from 7 to 12, the shift towards the alkaline area is not a mandatory condition and can be adjusted by adding ammonia or organic base.

Special thickeners (for example, polymer gels based on polyacrylates, (co)polymers of vinyl alcohol, ethers of cellulose, gelatin, agar-agar) can be added to the composition for the purpose of convenience of use.

The method of application of the claimed compositions is traditional and includes the following. The said composition is applied to dry or wet hair, left to work for 20 to 30 minutes for the purpose of activation and for 10 to 40 minutes for the purpose of toning, then the hair is rinsed and dried. There is no need in the subsequent treatment of the hair with a hair rinse, balm, or conditioner, because the claimed composition improves the hair condition (makes it more vivid, adds luster, makes the color more intensive and the hair—silky and well-groomed).

After being treated with the composition the hair can be submitted to any of the traditional hairdressers' procedures, e.g. cutting, styling, or dressing.

The potentialities of PFOC are not limited to the application examples mentioned above. Other properties of the emulsion—namely, to penetrate inside the hair and the capability of the particles of the dispersed phase (containing the above-mentioned PFOC in the outer layer) to break down inside the hair on contact with a melanin pigment grain,—served as a base for the authors of the invention, who suggested to use the reverse PFOC emulsions (of “water-in-oil” type) for an accelerated, less damaging and deeper coloring and bleaching of hair by traditional means.

The known bleaching agents, as it was noted before, consist, as a rule, of the ammonia solution of HP or other, more powerful oxidizers,—for example, persulfates of alkali or alkali-earth metals, or, which is much more seldom, of reducing agents.

Results of the bleaching of hair with an ammonia solution of HP and reverse emulsion of said solution in perfluorodecalin are given in Table 6.*

| No. of | | | | | Bleaching, |
|---|---|---|---|---|---|
| sample | Composition | SAA (surfactant) | PFD, % | HP, % | shade |
| 49 | Traditional solution | — | — | 4 | 0.6 ± 0.2 |
| 50 | without SAA | — | — | 10 | 2.2 ± 0.4 |
| | (surfactant) | | | | |
| 51 | Traditional solution | Chromoxane (thymaxol or | — | 4 | 1.6 ± 0.2 |
| | with SAA | proxanol) | | | |
| 52 | (surfactant) | Chromoxane (thymaxol or | — | 10 | 2.5 ± 0.3 |
| | | proxanol) | | | |
| 53 | Reverse emulsion | Chromoxane | 83.6 | 4 | 4 |
| 54 | | Chromoxane | 64 | 4 | 4 |
| 55 | | Chromoxane | 64 | 10 | 4.3 |
| 56 | Direct emulsion | Chromoxane | 16 | 4 | 1.7 |
| 57 | | Chromoxane | 11 | 10 | 2 |

*Hair color - Black-1; temperature 40° C.; pH - 9.5 (AH); treatment duration - 30 minutes.

It is evident from Table 6 that dispersion of traditional bleaching system in the PFOC claimed herein results in approximately two-fold increase of the bleaching system action efficiency (2.5 times increase for 4% HP (samples 53 and 54) and 1.7 times for 10% HP (sample 55)). The efficiency of action of direct emulsions (PFD in the ammonia solution of HP) of the same composition (samples 56 and 57) is the same as the one of traditional solutions. Moreover, in addition to an increase in the efficiency of action, the employment of reverse emulsions of hair melanin oxidizer in the claimed PFOC results in less damaging effect on hair in the course of bleaching (lower degree of hair cracking and hair felting). Lower degree of aggressiveness of claimed compositions represents the result of not only reduced content of HP in these compositions, but also at least four-fold reduction in the alkaline component concentration (Table 8).

Bleaching of other keratin-containing fibers (e.g. natural fur, wool) by the claimed composition proceeds in the same manner as hair bleaching, but it takes a shorter period of time (as a rule, 10-15 minutes).

TABLE 7
Comparison of bleaching levels and degree of damage to hair
when using L'Oreal compositions and reverse emulsions of
the ammonia solution of HP in PFD*

| | | Bleaching | | Number of | Mean degree | |
|---|---|---|---|---|---|---|
| | | conditions | | different | of bleaching | Hair damage |
| | | HP, % | | types of | after a period | index² after a |
| No. | Composition | by weight | pH | hair¹ | of 30 min. | period of 30 min. |
| 58 | Emulsion | 4 | 10 | 4 | 2.4 | — |
| | contains 43% | | | | | |
| | (by volume) of | | | | | |
| | PFD | | | | | |
| 59 | Emulsion | 6 | 10 | 4 | 2.9 | 0 |
| | contains 64% | | | | | |
| | (by volume) of | | | | | |
| | PFD | | | | | |
| 60 | Emulsion | 4 | 10 | 5 | 2.8 | 0.4 |
| | contains 75% | | | | | |
| | (by volume) of | | | | | |
| | PFD | | | | | |
| 61 | Emulsion | 2 | 10 | 4 | 1.2 | 0 |
| | contains 76% | | | | | |
| | (by volume) of | | | | | |
| | PFD | | | | | |
| 62 | Majiblond 913 | 9 | 10 | 3 | 1.4 | 1 |
| | L'Oreal | | | | | |
| 63 | Platifiz L'Oreal | 4.5³ | >12 | 4 | 2.9 | 1.3 |
| *Temperature - 30° C., treatment duration - 30 minutes. | | | | | | |
| ¹Black or reddish hair were bleached. | | | | | | |
| ²Each bleached hair sample was inspected on three kinds of damage to hair - namely, hair cracking, loss of hair elasticity and felting of the hair. Then all three changes in hair condition were summed for each hair sample. For example, if hair cracking and felting of the hair were observed for a certain hair sample, | | | | | | |
| #then an individual hair damage index is equal to 2. The hair damage index for a group was calculated by dividing the sum of individual indices into the number of samples tested. | | | | | | |

³Other oxidizers (persulfates) enter into the composition in addition to HP.

Beside HP, other oxidizers or reducing agents for melanin can be used in the claimed composition, with the same result gained. Thickeners, stabilizers, aromatic substances and so forth (both in the form aqueous solution and suspensions) may serve as components of the composition (see Table 8).

TABLE 8
Results of interaction between combined (filled) bleaching
compositions and hair of Blonde-5 color*

| | Content in | | Composition of aqueous phase | | | | | | |
|---|---|---|---|---|---|---|---|---|---|
| No. | the emulsion, | | HP, % | AH, | | | Bleaching, | | |
| of | % by mass | | by | % by | | Thickener | shades/time: | | |
| example | PFD | HP | mass | mass | Thickener | concentration, % | 10 min | 30 min | 60 min |
| 64 | 0 | 0 | 10.4 | 0.38 | Methyl cellulose | 1.3 | 3 | 3 | 3 |
| 65 | 60 | 4.2 | 10.4 | 0.15 | Methyl cellulose | 0.6 | 3.5 | 4.5 | 6.5 |
| 66 | 0 | 0 | 10.4 | 0.38 | Thymaxol | 2.6 | 2 | 2 | 4 |
| 67 | 60 | 4.2 | 10.4 | 0.15 | Thymaxol | 1.2 | 2 | 4 | 4.5 |
| 68 | 60 | 4 | 10 | 0.15 | — | 0 | 3.5 | 3.5 | 4 |
| 69 | 67 | 4 | 12.1 | 0.12 | — | 0 | 2 | 3 | 4.5 |
| 70 | 78 | 4.5 | 20.1 | 0.09 | — | 0 | 2.5 | 4.5 | 5 |
| 71 | 86 | 4 | 27.4 | 0.07 | — | 0 | 3.5 | 4 | 5 |

| | | | | | | | | |
|---|---|---|---|---|---|---|---|---|
| 71-a | 0 | 0 | 11 | 0.7 | 12% Oxygent from L'Oreal | 3.5 | 3.5 | 4.5 |
| 71-6 | 60 | 4.8 | 12 | 0.5 | 12% Oxygent from L'Oreal | 3.5 | 5.5 | 6.8 |

*At a temperature of 30° C., pH value - 10, with chromoxane (0.02%) used as surfactant. Viscosity of reverse emulsions with thickeners - from 7 to 10 poise.

Thus, a composition for bleaching keratin-containing fibers being a reverse “water-in-oil” emulsion of the aqueous solution or suspension of a bleaching agent in a perfluoroorganic compound is claimed as the third invention in the claimed group of inventions. The perfluoroorganic compound may be picked from the variety of the above-mentoned PFOC-solvents for melanin. The proportion of the perfluororganic compound phase in the above-mentoned two-phase system lies within 60-90% by mass. Both oxidizers (mainly, hydrogen peroxide) and reducing agents may serve as bleaching agent. The concentration of the bleaching agent in the aqueous phase may reach 45%, the value of pH of the aqueous phase varying over a broad range of 3.5 to 14.0. The shift to the alkaline area can be attained by adding ammonium hydroxide or organic base. It should be pointed out that it is desirable to use emulsions with pH over 11 only for hair coloring. The particle size in the mentioned two-phase system is 0.1-5 μm (mainly, 0.1-0.5 μm). Dispersions are stabilized with suitable surfactants (SAA), for instance, low-molecular (fluorine-containing SAA (chromoxane), or polymer (proxanol, thymaxol)). The proportion of SAA lies within 0.01-10% by mass.

Besides known factors (namely, oxidizer concentration, concentration of alkali agent and phase composition of the emulsion (Tables 6, 7 and 8), the size of a drop of dispersed oxidizer also influences the rate of bleaching. A three-fold decrease in the average size of drops results in 1.5-2-fold reduction in the effectiveness of bleaching (when bleaching is conducted through the use of emulsions containing from 43 to 74% (by volume) of PFOC. The same degree of reduction in the effectiveness of bleaching is observed when using a single-phase system (i.e. when using solutions—see Table 6).

An oxidizer based on HP (which is a component of dyes for oxidation dyeing and which includes thickeners and stabilizers) can be used for the preparation of bleaching emulsions. This oxidizer is produced by different companies.

The method of application of the claimed compositions is traditional and includes the following. The agent is applied to dry or wet hair, left to work for 10 to 30 minutes (predominantly, 10 minutes) then the hair is rinsed with water and dried. There is no need in the subsequent treatment of the hair with a hair rinse, balm, or conditioner, because the above-mentioned compositions retain the hair condition and exert conditioning effect. After being treated with the composition, the hair can be submitted to any of the traditional hairdressers procedures, e.g. cutting, styling, or dressing.

It is known that compositions most widely used for hair coloring are the so-called two-component oxidation colorants, being a mixture of water-soluble oxidation dye precursors (substituted phenols and/or aromatic amines) and the ammonia solution of HP. To compare the effectiveness of the traditional compositions for oxidation coloring and the reverse emulsions of these compositions in PFOC, a time index of attaining the similar intensity of coloring was used (Table 9).

TABLE 9
The effectiveness of hair coloring (producing hair color Blonde-1)
with reverse emulsions of the aqueous solution of an oxidation
colorant (OC) in perfluorodecalin*

| | | Oxidation | Dyeing | Time to | | | |
|---|---|---|---|---|---|---|---|
| | Hair color | dye | agent | produce the | | | |
| No. | attained | precursor | concentration, % | color, min | HP, % | AH, % | PFD, % |
| 72 | Dark brown | PPD | 0.1 | 30 | 0.63 | 0.13 | 0 |
| 73 | | | | 3 | 0.16 | 0.03 | 60 |
| 74 | Dark blue-black | PPD | 0.1 | 50 | 0.16 | 0.03 | 60 |
| 75 | | | | 15 | 15 | 0.13 | 0 |
| 76 | Chestnut | AP | 0.2 | 20 | 0.04 | 0.01 | 60 |
| 77 | | | | 40 | 1.5 | 0.8 | 0 |

*The process takes place at a temperature of 30° C.; SAA - thymaxol (0.2%).

From Table 9 it can be seen that using reverse emulsions of traditional oxidation agents in PFOC allows a 10-fold reduction of the coloring time and a 4-fold reduction of the concentration of components (HP and AH) aggressive to hair, with the same final result (see examples 72 and 73), or to shorten the time twice over, with a 40-fold reduction in the HP concentration and 80-fold reduction in ammonia concentration, to achieve the same final result (see examples 76 and 77). With a 100-fold reduction in HP concentration and 4-fold reduction in ammonia concentration (see examples 75 and 74) the coloring time, when the reverse emulsion is used, grows only to 40 minutes, which is a very high result, as compared to the known agents.

The reverse aqueous emulsions of natural colorants and low-toxicity colorants (for instance, of colorants used for dyeing textiles) in the indicated PFOC can also be used for hair dyeing.

Emulsification in PFOC of a mixture of oxidation colorants (produced by different companies in the form of compositions) that, in addition to oxidation colorant, contains solid finely dispersed fillers, SAA and ammonia, also results in the reduction in the concentration of HP and amount of dye paste required to obtain the same color (Table 10).

TABLE 10
The effect from emulsification in PFOC of a mixture of oxidation
colorants (OC) produced by L'Oreal for bleaching the hair
(Majiblond-937) and dyeing the hair to brown color (Majirel-4)*

| | | | Composition, % by | | | | |
|---|---|---|---|---|---|---|---|
| | | | mass | | | Time required to | |
| | | | | | Paste | obtain the uniform | |
| | Industrial mixture | | | | with | color | |
| | of oxidation dye | | | | oxidation | | Time, |
| No. | precursors | Oxidizer | PFD | HP | on dye | Color | min |
| 78 | Majirel-4 | HP solution, | 60 | 0.35 | 15 | Black/brown | 30 |
| | | pH = 10 | | | | | |
| 79 | | Oxygent-6% | 0 | 3.6 | 40 | | 30 |
| | | produced by | | | | | |
| | | L'Oreal | | | | | |
| 80 | Majiblond-913 | HP solution, | 60 | 2.9 | 8 | Light-light | 10 |
| | | pH = 10 | | | | brown | |
| 81 | | Oxygent-12% | 0 | 9 | 25 | | 30 |
| | | produced by | | | | | |
| | | L'Oreal | | | | | |

*Hair color - Blonde-5. Temperature - 30° C. SAA (chromoxane) −0.02%.

Emulsification of paste for dyeing to brown color in PFD enables one to obtain the same color for the same time period with the amount of dyeing agent being reduced three-fold and the amount of HP being reduced ten-fold. This significantly reduces the damage to hair keratin and skin of human head, as well as potential risk of skin irritation and allergies. Emulsification of bleaching paste in PFD allows a three-fold reduction of the coloring time with the amounts of dyeing agents and oxidizer being reduced three-fold.

Dyeing of other keratin-containing fibers with the composition claimed herein (e.g. natural fur, wool, plumes) proceeds in a manner similar to hair bleaching, but for a much shorter period of time (as a rule, no longer than 15 minutes).

It can be suggested that the reason for the effectiveness of using traditional coloring and bleaching compositions in the form of the reverse emulsion in the PFOC lies in the targeted delivery of reagents to melanin grains in the form of disperse particles, and in the breakdown of these particles with the absorption of the PFOC (melanin solvent) contained in the external layer of the particles on contact with the pigment inside the hair.

Thus, an agent for coloring keratin-containing fibers being a reverse “water-in-oil”-type emulsion of the aqueous solution or suspension of a colorant in a perfluoroorganic compound is claimed as the fourth invention in the claimed group of inventions. The perfluoroorganic compound may be picked from the variety of the above-mentioned PFOC-solvents for melanin. The proportion of the PFOC phase in the above-mentioned two-phase system is equal to 60-90% by mass. Oxidation dyeing agents, natural dyeing agents, non-toxic dyeing agents or the mixtures thereof can be taken as colorants. The concentration of the colorant or bleaching agent in the aqueous phase may reach 45%, pH of the aqueous phase varying over a broad range of 3.5 to 12.0. The shift to the alkaline area can be effected by adding ammonia hydroxide or organic base. The particle size in the mentioned two-phase system is 0.1-5 μm, predominantly—0.1-0.5 μm. Dispersions are stabilized with surfactants (SAA), for instance, low-molecular (fluorine-containing SAA (chromoxane), or polymer SAA (proxanol, thymaxol). The proportion of surfactant lies within 0.01-10% by mass.

The method of application of the claimed compositions is traditional and includes the following. The agent is applied to dry or wet hair, left to work for 10 to 40 minutes (predominantly, 20 minutes) then the hair is rinsed with water and dried. There is no need in the subsequent treatment of the hair with a hair rinse, balm, or conditioner, because the above-mentioned compositions retain the hair condition at the same level. After being treated with the composition, the hair can be submitted to any of the traditional hairdressers' procedures, e.g. cutting, styling, or dressing.

The claimed group of inventions: the solvent for melanin of keratin-containing fibers, a PFOC being used as such; the composition for activating the hair before bleaching or coloring, compositions for hair tinting, compositions for covering gray hair up (without using a dyeing agent), compositions for conditioning the hair, and the composition for bleaching and coloring keratin-containing fibers, form a single inventive design.

The above-described combination of essential features of each invention of the claimed group of inventions renders a technological result—quick and delicate hair treatment regardless of treatment type (activation, tinting, conditioning, bleaching or coloring). The attainment of the desired level of coloring is not accompanied by damage to hair structure. This is achieved through the new mechanism of selective neutralization of the hair melanin. Varying the PFOC concentration itself, apart from the added colorants, makes it possible to obtain hair tinting (more vivid or darkened natural color). PFOCs do not destroy keratin in their path, and therefore, are not spent uselessly. Unique is the effect of enhanced hair color for graying hair: there appears an effect of fewer gray hair. First yellowing was observed (after 10 minutes of contact), and then turning to brown (after 20 minutes of contact) in only gray hairs in the strand (see Table 4). Another additional technological result consists in conditioning of the hair (emergence of waviness) after 3-5 applications of the composition claimed herein. Even one (20 minutes long) application of the composition resulted in enhanced hair luster, better elasticity of dry and brittle hair and the effect of “fullness” with too soft and thin hair (without making the hair “heavier” in appearance, which is characteristic for surface-type conditioners).

The effect of the direct water emulsions with the claimed PFOC described in Table 3, unlike that of the known conditioning tinting composition—namely, tinting foam Wella Color Mousse,—does not deteriorate after washing with hot water (with shampoo Pantene Pro-V from P&G Blois), while the initial tint obtained with Wella foam gets twice as weak.

Therefore the necessity to use expensive repairing cosmetics or medications for the hair after coloring is eliminated.

The process of implementation of the compositions claimed herein can be controlled over a multitude of parameters and can be quickly and easily stopped when the desired degree of hair color modification is reached.

As an additional technological result, the safety of the claimed invention for the staff of hairdressers' facilities and their clients can be mentioned. This is so because PFOC are non-toxic, non-aggressive and don't cause allergies.

The invention claimed herein resembles the known oxidation method of hair dyeing in one thing only—the target of both is melanin neutralization. The nature of the claimed solvent, the type of dissolution, the solvent carrier, the compositions of emulsions and the results of the application of the claimed preparations are original. Thus, by the majority of essential features (except for the colorant and the bleaching agent in hair coloring or bleaching preparations) the claimed invention is different from the known method.

The analysis of the known level of technology did not reveal any solution fully coincident in the combination of essential features with the invention claimed herein.

As was indicated above, some PFOC serve as a base for blood substitutes. For example, the known ready-made blood substitute preparation with gas-transfer functions, “Perftoran”, is an aqueous emulsion of perfluorodecalin stabilized with phospholipins or proxanol [S. I. Vorobyov et al. A Comparative Study of Certain Perfluorocarbon Emulsions.—In: Fiziologicheskaya aktivnost' ftorsoderzhashchikh soyedineniy (in Russian).—Pushchino, 1995, pp.33-41]. However, this preparation has never been used for dissolution of melanin, or as a component of compositions for hair coloring.

Other areas of PFOC application, as organic solvents in particular, are not known.

It is necessary to emphasize that the capability of PFOC to dissolve melanin and modify the natural hair color was discovered by the authors of the invention by accident, and in order to explain this phenomenon as a capability of PFOC to arrange the state of aggregation of the pigment grains in the hair in an orderly fashion the authors carried out specific original research.

The analysis of the level of technology, therefore, supports the novel character of the proposition.

Only the combination of essential features of the invention claimed herein makes it possible to achieve the above-mentioned technological result. Up to these days it is believed that the principal application for PFOC is artificial blood substitutes. The original property of PFOC to dissolve melanin, quickly and producing colorless solutions at that, is, undoubtedly, not obvious. Unexpected were also the results of successful production of stable PFOC emulsions, capable of penetrating into the hair and dissolve melanin according to the mechanism of particle aggregation over a broad range, which by itself makes the natural hair color more vivid. The process of hair color modification becomes quick and gentle on the hair. More than that, the invention claimed herein facilitates the conditioning of the hair, which has practically never been observed with hair dyeing. It was impossible to presuppose that PFOC would be compatible with bleaching agents and dyes. The more so, it was impossible to imagine beforehand that the process under development would lead to a practicable result. Thus, a new functional dependence “composition-property” was implemented in the claimed invention. This serves to assert the correspondence of the proposed invention to the protection condition of “non-obviousness”.

Examples of Practical Implementation of the Invention are Given in Tables 2-10.

Non-living strands of natural hair (10 cm long) of colors indicated in Tables were used for treatment in our tests.

Direct and reverse emulsions were produced in an ultrasonic disperser operating in the following mode: power—(50-500) W, frequency—(22.5-40) kHz, cure time—2-5 min with concentrator area being within (0.5-2) cm². The size of emulsion particles was determined by the optical microscopy method (reverse emulsions) or nephelometry method (direct emulsions). Besides, direct emulsions of PFD stabilized with HP produced according to Temporary Pharmaceutical Regulations 42-25-76-99 were used.

The measurements of hair color were taken using the known “blind” method of expert evaluation by two experts. Original and treated strands of hair were placed in a monotonous row, and the samples differing by one or half a shade were noted. The samples between those, in dependence of their number and difference from one another, got a rating of 0.2 or 0.1 points, which was added (subtracted) from the indexes determined for the reference samples.

Solvent of Melanin

Melanin extracted from the mycelium and spores of the fungus Aspergillus niger was used to demonstrate new application of PFOC as solvent for melanin. Based on data of gel-permeation chromatography and results of component analysis, said model melanin is a low-molecular fraction (with MM 39,000 Da) of poly-5,6-dihydroxyindol-2-carboxylic acid. Data on solubility of model melanin in PFOC are given in examples 10-14 in Table 1.

A Composition for Activating the Hair Before Bleaching or Dyeing, for Hair Coloring, for Hair Toning for Covering Gray Hair up Without Using Dyeing Agents and for Hair Conditioning.

Examples from Table 2 (Activation of the Hair):

Aqueous solutions of HP containing 0.02% of chromoxane and 0.2% of AH (pH=11) were used as compositions for hair bleaching after activation.

### EXAMPLE 14

Bleaching of hair by traditional composition based on HP without preliminary activation. Bleaching of three strands of hair of Dark Brown-5 color was conducted using the following bleaching composition−6% aqueous solution of HP at a temperature of 30° C.:

the first strand of hair—during 10 minutes,

the second strand of hair—during 30 minutes,

the third strand of hair—during 60 minutes.

Upon the completion of bleaching the hair were rinsed with water and dried after which the degree of bleaching was determined which turned out to be as follows:

the first strand of hair—by 1.3 shade,

the second strand of hair—by 1.3 shade,

the third strand of hair—by 1.7 shade.

It should be pointed out that hair in the second strand became dull and hair in the third strand became brittle.

### EXAMPLE 15

### Bleaching of hair by traditional composition based on 6% HP with preliminary activation of hair with direct aqueous emulsion of PFOC.

For the purpose of hair activation, a ready-made emulsion of Perftoran (prepared according to Temporary Pharmaceutical Regulations 42-2576-99) that contained 16.2% by mass of PFD with the average diameter of particles being equal to 0.06 μm was concentrated by centrifuging (3000 rotations per second) until the content of PFD was equal to 27% with the average diameter of particles being equal to 0.09 μm. Three strands of hair of Dark Brown-5 color were placed into 20 ml of emulsion and kept there for a period of 30 minutes at a temperature of 40° C. Then the hair were rinsed with water and dried. Then after 24-hour period the strands were treated with emulsion once again in the above-indicated mode. The hair in all three strands acquired more vivid and bright appearance as compared to initial appearance.

Bleaching of three strands of hair of Dark Brown-5 color (treated with emulsion in advance) was conducted similarly to the procedure in example 14—i.e. using the following bleaching composition−6% aqueous solution of HP at a temperature of 30° C.:

the first strand of hair—during 10 minutes,

the second strand of hair—during 30 minutes,

the third strand of hair—during 60 minutes.

Upon the completion of bleaching, the hair were rinsed with water and dried after which the degree of bleaching was determined which turned out to be as follows:

the first strand of hair—by 1.5 shade,

the second strand of hair—by 2.0 shade,

the third strand of hair—by 2.0 shade.

The hair in all strands remained elastic and bright/lustrous after the bleaching procedure.

### EXAMPLE 16

the test was conducted according to the procedure of example 14 with strands of hair of Light Brown-2 color.

### EXAMPLE 17

the test was conducted according to the procedure of example 15 with strands of hair of Light Brown-2 color.

### EXAMPLE 18

the test was conducted according to the procedure of example 17, but preliminary activation was conducted for a period of 20 minutes at a temperature of 30° C.

Comparison of bleaching results in examples 16-18 gives an idea of the rate of diffusion of PFOC particles and rate of PFOC interaction with the melanin of hair.

### EXAMPLE 19

the test was conducted according to the procedure of example 14; the hair were bleached with 4% HP.

### EXAMPLE 20

the test was conducted according to the procedure of example 15; the hair were bleached with 4% HP.

### EXAMPLE 21

the test was conducted according to the procedure of example 16; the hair were bleached with 4% HP.

### EXAMPLE 22

the test was conducted according to the procedure of example 17; the hair were bleached with 4% HP.

### EXAMPLE 23

the test was conducted according to the procedure of example 18; the hair were bleached with 4% HP.

Examples From Table 3 (Hair Toning):

### Example 24

### Toning of Hair with Direct Aqueous Emulsion of PFOC

The composition was prepared as follows: 1 part (by mass) of egg lecithin (SAA) in the form of 2.2% aqueous colloid solution was diluted with water (74.5 parts by mass) and then 14.5 parts by mass of perfluorodecalin (PFOC) were added. Direct aqueous emulsion of perfluorodecalin was produced in an ultrasonic disperser operating in the following mode: power—500 W, frequency—22.5 kHz, cure time—5 min with concentrator area being 1 cm². The average size of particles in the emulsion was 0.15 μm, pH of dispersed aqueous phase was 9 (this value was obtained after adding aqua ammonia to the finished emulsion).

Then produced emulsion was applied on a strand of hair of Black-2 color and the hair were subjected to the action of the emulsion for a period of 30 minutes at a temperature of 40° C. Then the hair were rinsed with water and dried. The hair in the strand acquired more vivid appearance and dark (darker by 0.5 shade) color as compared to initial appearance and color. The hair remained elastic and silky. Reduction in the time of hair treatment with emulsion to 15 minutes gave identical results.

### EXAMPLE 25

46 parts by mass of 0.11% aqueous solution of chromoxane were added to 54 parts by mass of tri(perfluoropropyl)amine. Then the mixture was dispersed in the same manner as in example 24. The result of toning hair of Black-2 color by alkalized (pH=9) emulsion is similar to result obtained in example 24.

### EXAMPLE 26

the test was conducted according to the procedure of example 25; highly viscous solvent of melanin—namely, PFOC MF-30/40— was used for emulsifying. EXAMPLES 27 AND 28

the tests were conducted according to the procedure of example 24, but on strands of hair of Blonde-2 color. In the course of test 28 the hair sample was treated with direct emulsion three times (with a break between successive treatments being within 48-72 hours).

### EXAMPLE 29

the test was conducted according to the procedure of example 27, but without adding AH to the emulsion (with neutral pH).

### EXAMPLE 30

the test was conducted according to the procedure of example 29 (with neutral pH), but on strands of hair of Blonde-2 color. 56 parts by mass of PFD and 44 parts by mass of 2.3% colloid solution of PL (phospholipine) in water was used for the purpose of dispersion.

Examples from Table 4 (Covering Gray Hair Up):

### EXAMPLE 31

Emulsion for grayish hair toning (covering gray hair up) was prepared as described in example 29. Then emulsion was alkalized to pH=10 and after that applied on two strands of hair (of Black-4 type) that had individual gray hairs. Strands of hair were treated with emulsion for a period of 15 or 30 minutes at a temperature of 40° C. The result was as follows: gray hairs in dark hair became inconspicuous, because gray hairs grew darker quite considerably. The hair acquired volume and turned silky (prior to treatment they were dry).

### EXAMPLE 32

### Composition for Toning Hair (Coarse, Dull and Poorly Combed Hair) with Quite a Considerable Share of Gray Hair (Over 50%) was Prepared as Described in Example 30.

Then produced emulsion was applied on strands of hair and the hair was subjected to the action of the emulsion for a period of 20, 30 and 40 minutes at a temperature of 40° C. Then the hair were rinsed with water and dried. The hair in the strands acquired more vivid appearance and darker (by 0.5 shade) color as compared to initial appearance and color. The share of gray hair in strands was reduced (however, grayishness was distinguishable, because gray hairs acquired brownish-yellow tint). The hair became elastic and silky. The second treatment of the strand already treated for 30 minutes resulted in darkening of hair by up to 1.1 shade, and grayishness became inconspicuous.

### EXAMPLE 33

the test was conducted according to the procedure of example 32 (on the hair of the same type), but the hair was treated with an emulsion of Perftoran (prepared according to Temporary Pharmaceutical Regulations 42-2576-99) stabilized with HP.

### EXAMPLE 34

the test was conducted according to the procedure of example 33. The hair was treated with an emulsion of Perftoran emulsion by centrifuging method (in the same manner as in example 15).

### EXAMPLE 35

the test was conducted according to the procedure of example 31 (on the hair of Black-5 type, with pH being neutral).

### EXAMPLE 36

the test was conducted according to the procedure of example 32 on samples of hair of Light Broun-1 color (samples of hair had up to 30% of gray hair). The hair was very dry and brittle (in terms of hair structure, samples of hair resembled hair after chemical permanent wave. Following one treatment and two treatments for 30 minutes, the hair got darker by half a shade, gray hair get covered up (gray hairs acquire yellowish color). The hair also get “moisturized” and it is easier to comb hair subjected to treatment.

Examples from Table 5 (Conditioning of the Hair):

### EXAMPLES 37 AND 43

### Washing of Untreated Hair With Shampoo

A strand of coarse hair of Black-5 color (50% of gray hair) was washed several times (six times with a break of 2-3 days) with a shampoo Pantene Pro-V Lively Color. After each washing session the hair were dried and (without combing) studied. The color of the hair has not changed. The hair acquired dryness following two washing sessions.

### EXAMPLES 40 AND 46

### Washing of Untreated Hair With Shampoo

A strand of hair of Light Brown-2 color (wavy and dry hair characterized by the “numbness” effect) was washed several times (six times with a break of 2-3 days) with a shampoo Pantene Pro-V Lively Color. After each washing session the hair were dried and (without combing) studied. The dryness of the hair has increased following the first washing session (and the hair became disentangled), and hair tangling was observed after the second washing session.

### EXAMPLE 38

### Washing of Hair Pretreated With SAA

A strand of coarse hair of Black-5 color (50% of gray hair) [identical to strand of hair from example 37] was treated with 1% colloid solution of PL (diameter of micelles—0.13 μm) for 20 minutes at a temperature of 40° C. Then this strand of hair was rinsed in water and dried (without combing prior to drying). The hair acquired the “unified styling effect” (but without visual effect of “increased weight of hair”). Besides, the hair turned darker by 0.5 shades. The treated strand of hair was then washed six times with a break of 2-3 days with a shampoo Pantene Pro-V Lively Color. After each washing session the hair were dried and (without combing) studied. The hair turned coarser after the first washing sessions, and the initial color of the hair was “restored” after the second washing session, and hair tangling was observed after the third washing session.

### EXAMPLE 39

### Washing of Hair Pretreated With Direct Aqueous Emulsion of PFOC.

A strand of coarse hair of Black-5 color (50% of gray hair) [identical to strands of hair from examples 37 and 38] was treated with an aqueous emulsion of PFOC in the same manner as described in example 32 from Table 4 for a period of 20 minutes. The hair in the strand acquired more “vivid” color and got darker by 0.9 shade (as compared to initial color). The gray hair acquired brownish color and became inconspicuous on the background of other hair. The hair became elastic, silky and slightly wavy. The treated strand of hair was then washed six times with a shampoo Pantene Pro-V Lively Color. After each washing session the hair were dried and (without combing prior to drying) studied. The gray hair showed up after six washing sessions, and the dryness of hair—after five washing sessions.

### EXAMPLE 41

the test was conducted according to the procedure of example 38, but on strands of auburn hair of Light Brown-2 type. The treatment with a colloid solution of PL for a period of 20 minutes resulted in darkening of hair by 0.8 shade and in enhancement of hair luster. The first washing session has restored the initial color of hair, and after the second washing session knotting of the hair was observed.

### EXAMPLE 42

the test was conducted according to the procedure of example 39, but on strands of auburn hair of Light Brown-2 type. The treatment with a PFD emulsion for a period of 20 minutes resulted in darkening of hair by 0.5 shade, in enhancement of hair luster and improvement of hair smoothness. Obtained color of hair (as well as all conditioning effects) was retained for a period of four washing sessions.

### EXAMPLE 44

the test was conducted according to the procedure of example 38, but the treatment of hair was performed with 8% solution of proxanol (stabilizer of Perftoran emulsion). The treatment resulted in darkening of hair by 0.5 shade, but gray hair were not colored properly and the effect of traditional conditioning accompanied by “visual effect of increased weight of hair” was observed. Darkening of the hair disappeared after the first washing session; the hair became dry after the second washing session, and severe felting of the hair was observed after the third washing session.

### EXAMPLE 45

the test was conducted according to the procedure of example 39. The hair were treated with a PFOC emulsion stabilized with proxanol (the emulsion similar to one employed in example 15 was used). Following the 20-minute treatment, the hair got darker by 0.6 shade, became glossy, gray hair got darker and straightened. The condition of the hair turned worse only after five washing sessions, and the changes in hair color were observed only after the sixth washing session.

### EXAMPLE 47

the test was conducted according to the procedure of example 44 on strands of Light Brown-2 hair. The treatment resulted in darkening of hair by 0.5 shade and in improvement of hair condition (the hair acquired the “unified styling effect”). After the first washing session the obtained effects were retained, but the initial color of hair was restored after the second washing session, and the hair became dry after the second washing session.

### EXAMPLE 48

the test was conducted according to the procedure of example 45 on strands of Light Brown-2 hair. The treatment resulted in darkening of hair by 0.2 shade and in improvement of hair condition (which was manifested in hair wetting, in the fact that the effect of hair cracking has disappeared, and in the fact that the hair looked like glossy and easily mobile volume). All the obtained effects were retained till after the fifth washing session.

Composition for Bleaching Keratin-Containing Fibers

Examples from Table 6 (Bleaching of the Hair):

### EXAMPLE 49

### Bleaching of the Hair with Traditional Composition Based on HP.

Bleaching of hair strand of Black-1 color was conducted using a bleaching composition (4% ammonia aqueous solution of HP with pH 9.5) for a period of 30 minutes at a temperature of 40° C. Upon the termination of bleaching, the hair was rinsed with water, dried and then the degree of bleaching was determined. The tests were conducted on four hair samples and the following bleaching results were obtained: 0.5 shade, 0.5 shade, 0.8 shade and 0.9 shade. On the average, the bleaching effect was equal to 0.6 shade. The hair became more dull.

### EXAMPLE 50

the test was conducted according to the procedure of example 49. The bleaching was carried out with 100% ammonia solution of HP. The tests were conducted on four hair samples and the following bleaching results were obtained: 2 shades, 2 shades, 2 shades and 2.7 shades. On the average, the bleaching effect (after 30 minute period of bleaching) was equal to 2.2 shades.

### EXAMPLE 51

### Bleaching of Hair With Traditional Composition Based on HP in the Presence of SAA

Bleaching of hair strand of Black-1 color was conducted using a bleaching composition (4% ammonia aqueous solution of HP with pH 9.5) in the presence of 0.01 parts by mass of potassium perfluoro-4-methyl-3,6-dioxaoctansulfonate (chromoxane, SAA) or in the presence of 0.2 parts by mass of Thimaxol or proxanol (polymer SAA) for a period of 30 minutes at a temperature of 40° C. Upon the termination of bleaching, the hair was rinsed with water and dried. The results of 16 tests were averaged (1.5; 1.5; 1.5; 1.5; 1.5; 1.5; 1.5; 1.7; 1.7; 1.7; 1.7; 1.7; 1.7; 2; 2; and 2 shades). On the average, the bleaching effect was equal to 1.6 shades. The hair became duller.

### EXAMPLE 52

the test was conducted according to the procedure of example 51. The bleaching was carried out with 10% HP in the ammonia solution of the same types of SAA. The tests were conducted on six hair samples and the following bleaching results were obtained: 2.5; 2.5; 2.6; 2.7; 2.7; and 2.8 shades. On the average, the bleaching effect was equal to 2.5 shades.

### EXAMPLE 53

### Bleaching of Hair with Reverse Emulsion of a Traditional Composition Based on HP in PFOC

17.3 parts (by mass) of an aqueous solution that contained 27.4% of HP and 0.08% of chromoxane (SAA) were added to 83.6 parts (by mass) of PFOC-perfluorodecalin. Reverse emulsion of an aqueous solution of HP in PFD was produced in an ultrasonic disperser operating in the following mode: power—500 W, frequency—22.5 kHz, cure time—5 min with concentrator area being 1 cm². The average size of particles in the emulsion was 0.15 μm. Then 0.8 parts (by mass) of 8% aqueous solution of ammonia was added and mixed with said emulsion. The value of pH of the emulsion was 10.

Bleaching of hair with produced emulsion was carried out in the same manner as bleaching with traditional solutions described in examples 49-52 (temperature-40° C., hair—of Black-1 type). After 30 minutes of bleaching, the strand of hair was extracted, rinsed with water, dried and then the degree of bleaching was determined (which was equal to 4 shades).

### EXAMPLE 54

the test was conducted according to the procedure of example 53, but the composition of emulsion was different. 37 parts (by mass) of water containing 10.1% of HP and 0.1% of chromoxane (SAA) were added to 63 parts (by mass) of PFD. The mixture was then dispersed (in the same manner as in example 53), after which ammonia was added to bring the value of pH to 10. Then a strand of hair of Black-1 type was bleached at a temperature of 40° C. After 30-minute bleaching the degree of bleaching was equal to 4 shades. The hair remained bright and elastic.

### EXAMPLE 55

the test was conducted according to the procedure of example 54, but the composition of the aqueous phase of the emulsion was different. 36 parts (by mass) of water containing 27.6% of HP and 0.1% of chromoxane (SAA) were added to 64 parts (by mass) of PFD. The mixture was then dispersed (in the same manner as in example 53), after which ammonia was added to bring the value of pH to 10. Then a strand of hair of Black-1 type was bleached at a temperature of 40° C. After 30-minute bleaching the degree of bleaching was equal to 4.3 shades. The hair remained bright and elastic.

### EXAMPLE 56

Bleaching of Hair with Direct Emulsion of PFD in a Traditional Composition Based on HP.

84.3 parts (by mass) of an aqueous solution containing 4.7% of HP and 1.5% of PL (SAA) were added to 15.6 parts (by mass) of PFD. The mixture was then dispersed, after which the value of pH was brought to be equal to 10. Then a strand of hair was bleached with produced emulsion (in the same manner as in example 53). After 30-minute bleaching the level of bleaching was equal to 1.7 shades.

### EXAMPLE 57

Bleaching of Hair with Direct Emulsion of PFD in a Traditional Composition Based on HP.

89 parts (by mass) of an aqueous solution containing 12% of HP and 0.04% of chromoxane were added to 11 parts (by mass) of PFD. The mixture was then dispersed, after which the value of pH was brought to be equal to 10. Then a strand of hair was bleached with produced emulsion (in the same manner as in example 53). After 30-minute bleaching the level of bleaching was equal to 2 shades.

### EXAMPLE 58

the test was conducted according to the procedure of example 54, but the composition of emulsion and bleaching conditions were different from those in example 54. 40 parts (by mass) of an aqueous solution containing 10% of HP and 0.5% of chromoxane were added to 60 parts (by mass) of PFD. The mixture was then dispersed (in the same manner as in example 54), after which the value of pH was brought to be equal to 10. Then four strands of hair (two strands of dark hair and two strands of red hair) were bleached. The strands of hair were bleached at a temperature of 30° C. After bleaching the strands for 30 minutes, the samples were rinsed with water and dried, after which the degree of bleaching for all hair strands was determined. It was equal to 2.5, 2.7, 2.7 and 1.6 shades. On the average, the level of bleaching was 2.4 shades. The hair remained bright and elastic after bleaching.

### EXAMPLE 59

the test was conducted according to the procedure of example 58, but the composition of emulsion was different from that in example 58. 22 parts (by mass) of an aqueous solution containing 27.4% of HP and 0.5% of chromoxane were added to 78 parts (by mass) of PFD. The produced mixture was then dispersed (in the same manner as in example 54), after which 0.1 parts (by mass) of AH was added to the mixture to bring the value of pH to 10. Then four strands of hair (two strands of dark hair and two strands of red hair) were bleached at a temperature of 30° C. After bleaching the strands for 30 minutes, the samples were rinsed with water and dried, after which the degree of bleaching for all hair strands was determined. It was equal to 1.8, 3.9, 3.2 and 2.5 shades. On the average, the degree of bleaching was 2.9 shades. The hair remained bright and elastic after bleaching, no hair cracking and knotting of the hair is observed.

### EXAMPLE 60

the test was conducted according to the procedure of example 58, but the composition of emulsion was different from that in example 58. 15 parts (by mass) of an aqueous solution containing 27.4% of HP and 0.5% of chromoxane were added to 85 parts (by mass) of PFD. The produced mixture was then dispersed (in the same manner as in example 54), after which 0.07 parts (by mass) of AH was added to the mixture to bring the value of pH to 10. Then four strands of hair (two strands of dark hair and two strands of red hair) were bleached at a temperature of 30° C. After bleaching the strands for 30 minutes, the samples were rinsed with water and dried, after which the degree of bleaching for all hair strands was determined. It was equal to 2.4, 3.9, 3.0 and 1.9 shades. On the average, the degree of bleaching was 2.8 shades. The hair remained bright and elastic after bleaching, no knotting of the hair is observed. Hair cracking is observed only in two samples.

### EXAMPLE 61

the test was conducted according to the procedure of example 60, but the composition of an aqueous phase was different from that in example 60. 22 parts (by mass) of an aqueous solution containing 15% of HP and 0.5% of chromoxane were added to 78 parts (by mass) of PFD. The produced mixture was then dispersed (in the same manner as in example 54), after which 0.06 parts (by mass) of AH was added to the mixture to bring the value of pH to 10. Then four strands of hair (two strands of dark hair and two strands of red hair) were bleached at a temperature of 30° C. After bleaching the strands for 30 minutes, the samples were rinsed with water and dried, after which the degree of bleaching for all hair strands was determined. It was equal to 0.7, 1.3, 1.5 and 1.3 shades. On the average, the degree of bleaching was 1.2 shades. The hair remained very elastic after bleaching, no hair cracking (i.e. split ends of hair) and knotting of the hair is observed.

### EXAMPLE 62

Bleaching of four strands of hair (of the same type as were used in examples 58-61) was conducted using a composition for professional bleaching of hair Majiblond-913 (produced by L'Oreal). The bleaching was conducted at a temperature of 30° C. according to operating instructions to said composition. After mixing the components, the value of pH of the bleaching paste was equal to 10, while the concentration of HP in the paste (calculated) was equal to 9%. The paste was applied on four strands of hair. After bleaching the strands for 30 minutes, they were rinsed with water and dried, after which the degree of bleaching for all hair strands was determined. It was equal to 1.5, 0.8, 1.5 and 1.8 shades. On the average, the degree of bleaching was 1.4 shades. The hair after bleaching was very dry and dull. Hair cracking (i.e. split ends of hair) was observed in two strands, felting of the hair was observed in two strands.

### EXAMPLE 63

Bleaching of four strands of hair (of the same type as were used in examples 58-61) was conducted using a composition for professional frosting of hair Platifiz (produced by L'Oreal). The bleaching was conducted at a temperature of 30° C. according to operating instructions to said composition. After mixing the components, the value of pH of the bleaching paste was over 10, while the concentration of HP in the paste (calculated) was equal to 4.5%. Besides HP, the paste includes other oxidizers (persulfates). The paste was applied on four strands of hair. After bleaching the strands for 30 minutes, they were rinsed with water and dried, after which the degree of bleaching for all hair strands was determined. It was equal to 2.2, 4, 2 and 3.5 shades, respectively. On the average, the degree of bleaching was 2.9 shades. The hair after bleaching was very dry and dull. Hair cracking (i.e. split ends of hair) was observed in two strands, felting of the hair was observed in two strands.

Examples from Table 8.

### EXAMPLE 64

30 parts (by mass) of an aqueous 34.8% solution of HP and 4 parts (by mass) of 0.5% chromoxane (SAA) were added to 66 parts (by mass) of methyl cellulose gel in water. The produced mixture was then dispersed in an ultrasonic disperser for a period of 3 s, after which aqua ammonia was added to the mixture to bring the value of pH to 10.5. Then produced paste was applied on three strands of hair of Blonde-5 type. The bleaching was conducted at a temperature of 30° C. for a period of 10 minutes, 30 minutes, and 60 minutes, respectively. Then the strands were extracted, rinsed with water and dried in the air, after which the degree of bleaching for all hair strands was determined by comparing said strands with initial hair samples.

### EXAMPLE 65

12 parts (by mass) of an aqueous 34.8% solution of HP and 0.02 parts (by mass) of solid chromoxane (SAA) were added to 28 parts (by mass) of methyl cellulose gel in water. The produced mixture was then dispersed in an ultrasonic disperser for a period of 3 s, after which 60 parts (by mass) of PFD (PFOC) was added to the mixture and the latter was dispersed for a period of 30⁻s in a disperser, the power of which was 50 W and concentrator was 1 cm² in diameter. Then a solution of aqua ammonia was added to produced paste-like emulsion to bring the value of pH to 10.5. The bleaching of the hair of Blonde-5 type was conducted in the same manner as described in example 64.

The composition of an aqueous phase of compositions described in examples 64 and 65 is the same, but the rate of bleaching and quality (condition) of bleached hair is better in the case when the emulsion was used.

### EXAMPLE 66

Gel-like paste was prepared in the same manner as described in example 64, but on the basis of 4% gel of thymaxole (SAA). The bleaching of the hair was conducted in the same manner as described in example 64.

### EXAMPLE 67

Paste-like reverse emulsion was prepared in the same manner as described in example 65, but 4% gel of thymaxole was used as a thickener. The bleaching of the hair was conducted in the same manner as described in example 64.

The composition of an aqueous phase of compositions described in examples 65 and 66 is the same, but the rate of bleaching and quality (condition) of bleached hair is better in the case when the emulsion was used.

### Example 68

Reverse emulsion was prepared in the same manner as described in example 53, but aqua ammonia was added to bring the value of pH to 10.5. The bleaching of the hair of Blonde-5 type was performed in this test. The strands of hair were bleached for a period of 10 minutes, 30 minutes, and 60 minutes, respectively. Then the strands were extracted from emulsion, rinsed with water and dried in the air, after which the degree of bleaching for all hair strands was determined.

### EXAMPLE 69

Reverse emulsions were prepared in the same manner as described in example 68, but with a different proportion of reagents. The bleaching of the hair was conducted in the same manner as described in example 68.

### EXAMPLE 70

Reverse emulsions were prepared in the same manner as described in example 68, but with a different proportion of reagents. The bleaching of the hair was conducted in the same manner as described in example 68.

### EXAMPLE 71

Reverse emulsions were prepared in the same manner as described in example 68, but with a different proportion of reagents. The bleaching of the hair was conducted in the same manner as described in example 68.

### EXAMPLE 71-a

The bleaching of the hair of Blonde-5 type was conducted in the same manner as in example 64 (after adding aqua ammonia to 12% Oxygent produced by L'Oreal to bring the value of pH to 10.5).

### EXAMPLE 71-b

Reverse emulsion was prepared in the same manner as described in example 65, but 12% Oxygent (produced by L'Oreal) was used as an aqueous phase. Following the dispersion of the paste in PFD, aqua ammonia was added to the thick dispersion (to bring the value of pH to 10.5), after which the bleaching process was conducted in the same manner as in example 68.

Composition for Coloring Keratin-Containing Fibers

Examples from Table 9 (Coloring of the Hair).

### EXAMPLE 72

### Dyeing of the Hair Using the Known Oxidation Coloring Agent Combined with an Aqueous Solution of HP with AH.

Dyeing of the hair of Blonde-1 type was conducted using an aqueous solution of the oxidation dye precursor AP (p-aminophenol) containing 0.1% (by mass) of dyeing agent, 0.63% (by mass) of HP and 0.13% (by mass) of AH (pH=9) at a temperature of 30° C. for a period of 30 minutes. Upon the termination of dyeing, the hair was rinsed with water, dried and then the degree of coloring was determined (dark-brown shade). The hair became dull.

### EXAMPLES 74, 76

these tests were conducted according to the procedure of example 72, but using different oxidation dye precursor (example 76) and different composition of the oxidizer (examples 74 and 76).

### EXAMPLE 73

Dyeing of the hair with reverse emulsion of the known oxidation dye precursor PA (p-aminophenol). Solvent for melanin—a PFOC-perfluorodecalin (PFD).

0.2 parts (by mass) of poly-N,N-dimethylaminoethyl metacrylate (thymaxol) (SAA) in the form of 18% aqueous gel were added to 39.8 parts (by mass) of an aqueous solution of oxidation dye OK-30 that contained 0.1% (by mass) of dyeing agent, 0.16% (by mass) of HP and 0.03% (by mass) of AH (pH=9). Then 60 parts (by mass) of perfluorodecalin (PFOC) were added to the solution produced at the previous step. The reverse emulsion of an ammonia aqueous solution of dyeing agent OK-30 in PFD was produced in an ultrasonic disperser operating in the following mode: power—50 W, frequency—22.5 kHz, cure time—1.5 min with concentrator area being 1 cm². The average size of particles in the emulsion was 0.15 μm. The value of pH of the dispersed medium was 9. The coloring of a strand of hair of Black-1 color was performed at a temperature of 30° C. using the produced reverse emulsion. Despite the fact that the content of HP in example 73 is four times lower than the content of HP in example 72 (0.63% by mass of HP), the dark-brown color of hair (identical to that attained in example 72) was produced after 3 minutes of dyeing. Upon the termination of dyeing, the hair was rinsed with water, dried and then the degree of coloring was determined (dark-brown). The hair in the strand remained elastic and lustrous.

### EXAMPLE 74

the test was conducted according to the procedure of example 73, but the content of HP was different (0.16%). After dyeing the hair for 50 minutes, the hair acquired deep blue-black color. The similar color is obtained after traditional dyeing (example 75) conducted for 15 minutes with the concentration of HP being 100 times higher.

### EXAMPLE 76

the test was conducted according to the procedure of example 73, but the content of HP was different (0.04%) and another oxidation due precursor (p-aminophenol) was used. After dyeing the hair for 20 minutes at a temperature of 30° C., the hair acquired deep golden-auburn color. The similar color is obtained after traditional dyeing (example 77) conducted for 40 minutes with the concentration of HP being 37 times higher.

### EXAMPLE 78

15 parts (by mass) of the Majirel-4 paste (containing oxidation dye precursors, AH, thickeners, color modifiers, stabilizers and so forth) were added to 60 parts (by mass) of PFD. The produced mixture was then emulsified in an ultrasonic disperser (power—50 W, area of wave guide cut-1 cm²) for a period of 2 minutes. Then 25 parts (by mass) of an aqueous 1.4% solution of HP was added to the produced thick paste, after which this mixture was emulsified for 1 minute (pH=10). Then produced paste was applied on the hair of Blonde-5 type for a period of 30 minutes at a temperature of 30° C. Then the hair was rinsed with water and dried. The color of hair was black-brown, and the hair looked shiny and moistened.

### EXAMPLE 79

Dyeing agent produced by L'Oreal for professional hair dyeing into brown color was prepared according to operating instructions for this dyeing agent. 60 parts (by mass) of Oxygent-6% were added to 40 parts (by mass) of this dyeing agent (in the form of paste), and the new composition was mixed until a homogenous mixture is produced. The calculated content of HP in the produced composition was 3.6%. Then produced paste was applied on the hair of Blonde-5 type for a period of 30 minutes at a temperature of 30° C. Then the hair was rinsed with water and dried. The color of hair was black-brown (similar to the color obtained in example 79).

### EXAMPLE 80

8 parts (by mass) of the Majiblond-913 paste (containing oxidation dye precursors, AH, thickeners, color modifiers, stabilizers and so forth) were added to 60 parts (by mass) of PFD. The produced mixture was then emulsified in an ultrasonic disperser (power—50 W, area of wave guide cut-1 cm²) for a period of 2 minutes. Then 32 parts (by mass) of an aqueous 9% solution of HP was added to the produced thick paste, after which this mixture was emulsified for 1 minute (pH=10). Then produced paste was applied on the hair of Blonde-5 type for a period of 30 minutes at a temperature of 30° C. Then the hair was rinsed with water and dried. The color of hair was ash-light-brown, and the hair looked shiny and moistened.

### EXAMPLE 81

Dyeing agent produced by L'Oreal for professional hair bleaching was prepared according to operating instructions for this dyeing agent. 75 parts (by mass) of Oxygent-12% were added to 25 parts (by mass) of this dyeing agent (in the form of paste), and the new composition was mixed until a homogenous mixture is produced. The calculated content of HP in the produced composition was 9%. Then produced paste was applied on the hair of Blonde-5 type for a period of 30 minutes at a temperature of 30° C. Then the hair was rinsed with water and dried. The color of hair was ash-light-brown (similar to the color obtained in example 80).

The practical implementation of the invention claimed herein is not limited to the above-described examples. The invention claimed herein expands to any keratin-containing fibers (in addition to hair)—namely, to natural fur, wool, plumes.

The results listed in Tables 2-10 testify that implementation of the invention claimed herein enables one to modify the color of hair of different intensity of natural color (black hair, auburn hair, light-brown hair) to a pre-specified degree of toning, while preserving the hair structure, natural luster of hair, hair curl and ability of natural hair to get styled. Since balms are not used, the hair retains their natural properties for the entire observation period (up to 6 months). After bleaching of hair, the residual yellowishness or reddishness of hair is either not observed at all (in the case when AH is not present in an emulsion) or almost inconspicuous, which is associated with the fact that a lower amount of HP (and, correspondingly, lower amount of ammonia) is used in the method suggested herein. In contrast to the proposed method, the disturbance of hair structure and loss of hair luster is observed in hair bleached by the known method through the use of the known composition (even in the case of pre-specified degree of bleaching by one shade). In order to impart “naturalness” to such hair, one has to use hair lacquer and to cut this hair in 6 months, because they begin contrasting quite sharply (brittleness, cracking/split ends) with newly grown hair. The use of reverse emulsions of the known dyeing agents and oxidizers in PFOC results in significant increase in coloring effectiveness. Using the invention claimed herein one could perform the dyeing of hair faster and under the conditions that are less damaging to natural hair.

Going beyond the limits of the claimed values of concentrations of agents that enter into the compositions will result either in the impossibility of solvent penetration into the hair and thus to the impossibility of the implementation of the invention claimed herein (in the case of going beyond the upper limit) or in the prolongation of hair coloring duration undesirable for clients (in the case of going beyond the lower limit).

1. At least one perfluoro-organic compound as a solvent for melanin of keratin-containing fibers.

2. The method of claim 1, wherein at least one perfluoro-organic compound is selected from the group consisting of perfluorinated hydrocarbon, perfluorinated tertiary amine, perfluorinated alcohol and perfluorinated ketone.

3. A composition for treating hair, the composition being an “oil in water” type emulsion having a dispersed phase comprising particles of at least one perfluoro-organic compound and a dispersion medium comprising water and an emulsifier in the proportion (% by mass): from 2% to 95% of the dispersed phase, from 0.01% to 10% of the emulsifier, and the rest being the dispersion medium, wherein the sizes of the particles do not exceed 0.5 μm.

4. The composition of claim 3, wherein the dispersion medium has a pH ranging from about 7 to about 12.

5. The composition of claim 4, further comprising an aqueous solution of ammonia or an organic base capable of altering the pH of the dispersion medium.

6. The composition of claim 3, wherein the emulsifier comprises a surfactant.

7. The composition as in claim 6, wherein the surfactant is a water insoluble surfactant comprising a phospholipid, or the surfactant is a polymeric surfactant comprising a water soluble compound selected from the group consisting of proxanol, thymaxol and methylcellulose, or the surfactant is a water soluble low-molecule ionogenic surfactant comprising chromoxane.

8. The composition of claim 3, further comprising a thickener.

9. A composition for changing color of keratin-containing fibers, the composition being a “water in oil” reverse emulsion having an aqueous dispersed phase containing a bleaching agent or a dyeing agent, and a dispersion medium comprising at least one perfluoro-organic compound and an emulsifier in the proportion (by % mass): from 60% to 90% of the dispersion medium, from 0.01% to 10% of the emulsifier, and the rest being the dispersion phase, wherein the sizes of the particles of the dispersed phase ranges from about 0.1 μm to 5 μm.

10. The composition of claim 9, wherein a concentration of the bleaching agent or the dyeing agent in the dispersed phase does not exceed 45%.

11. The composition of claim 9, wherein the bleaching agent is an oxidizer.

12. The composition of claim 11, wherein the oxidizer is hydrogen peroxide or a compound selected from the group consisting of persulfates, perborates, periodites, and perclorates of alkali or alkali-earth metals.

13. The composition of claim 9, wherein the bleaching agent is a reducing agent.

14. The composition of claim 13, wherein the reducing agent is selected from the group consisting of sulfites of alkali metals, hydrosulfites of alkali metals, sulfites of alkali-earth metals, hydrosulfites of alkali-earth metals, thioacids, thiols, or oxalic acid.

15. The composition of claim 9, wherein the bleaching agent comprises a thickener.

16. The composition of claim 15, wherein the thickener is in the form of aqueous solution, gel or suspension.

17. The composition of claim 9, wherein the bleaching agent or the dyeing agent contain a stabilizer.

18. The composition of claim 17, wherein the stabilizer is in the form of aqueous solution, gel or suspension.

19. The composition of claim 9, wherein the bleaching agent contains an aromatizing substance.

20. The composition of claim 19, wherein the aromatizing substance is in the form of aqueous solution, gel or suspension.

21. The composition of claim 9, wherein the dyeing agent comprises a mixture is dyes.

22. The composition of claim 9, wherein the dyeing agent is an oxidizing dye, a natural dye, or a non-toxic textile dye.

23. The composition of claim 9, wherein the dyeing agent is in the form of aqueous solution, suspension or paste.

24. The composition of claim 9, wherein the dyeing agent further comprises a surfactant.

25. The composition of claim 9, wherein the dyeing agent further comprises an aqueous solution of ammonia.

26. The composition of claim 9, wherein the dispersed medium has a pH ranging from about 3.5 to about 14.

27. The composition of claim 26, further comprising an aqueous solution of ammonia or an organic base capable of altering the pH of the dispersion medium.

28. The composition of claim 9, wherein the emulsifier comprises a surfactant.

29. The composition of claim 28, wherein the surfactant is a water insoluble surfactant comprising a phospholipid, or the surfactant is a polymeric surfactant comprising a water soluble compound selected from the group consisting of proxanol, thymaxol and methylcellulose, or the surfactant is a water soluble low-molecule ionogenic surfactant comprising chromoxane.
